(19) United States
(12) Patent Application Publication
(10) Pub. No.: US 2019/0112577 A1
(43) Pub. Date: Apr. 18, 2019

(54) EX VIVO EXPANSION METHOD FOR CORD BLOOD NK CELL, AND KIT AND APPLICATION THEREOF

(71) Applicant: BEIJING JING-MENG STEM CELL TECHNOLOGY CO., LTD., Beijing (CN)

(72) Inventors: Xiaoyun WU, Beijing (CN); Xuemin LIU, Beijing (CN); Huiyan KANG, Beijing (CN); Yunhong WANG, Beijing (CN); Xiaoxiang FAN, Beijing (CN); Zhigang LI, Beijing (CN); Wei DING, Beijing (CN); Lihua WU, Beijing (CN); Jinyan LI, Beijing (CN); Dianfu SHEN, Beijing (CN); Huizhen ZHANG, Beijing (CN)

(21) Appl. No.: 16/090,258

(22) PCT Filed: May 6, 2016
(86) PCT No.: PCT/CN2016/081198

§ 371 Date: Sep. 30, 2018

(30) Foreign Application Priority Data
Apr. 7, 2016 (CN) 201610214351.1

(51) Int. Cl. C12N 5/0783

(52) U.S. Cl. CPC C12N 5/0646; C12N 2501/2302; C12N 2501/999; C12N 2501/2318; C12N 2501/2315

(57) ABSTRACT

Provides an ex vivo expansion method for a cord blood NK cell, and a kit and application thereof. The ex vivo expansion method for the cord blood NK cell comprises: separating a cord blood monocyte; activating, culturing, and proliferation; and culturing of the cord blood NK cell. The invention does not require cell sorting and does not require a trophoblast. A reagent in the kit does not contain animal-based component. The invention provides a high production volume of the NK cell. The cord blood NK cell obtained has at least 90% purity, a total cell count of up to 10¹⁰⁻¹¹ per part of cord blood, and can kill a tumor cell effectively.

### TECHNICAL FIELD

The present disclosure relates to the field of cell engineering technology, in particular to an ex vivo expansion method for cord blood NK cells and an ex vivo expansion kit for the cord blood NK cells, and further to applications of the cord blood NK cells in preparing tumor immunotherapy drugs and in tumor immunotherapy.

### BACKGROUND

Nature killer (NK) cells are a third type of lymphocytes in addition to T cells and B cells, and they also have different cell morphology from the T cells and B cells. The NK cells are large granular lymphocytes in cytoplasm and containing azurophilic granules, and are widely found in lymphoid organs and peripheral tissues. The NK cells account for 5% to 10% of the total number of lymphocytes in normal peripheral blood, account for 1% to 2% of the total number of lymphocytes in the spleen, and also exist in lymph nodes and bone marrow. As an important constituent part of the human body's natural immunity, the NK cells are important immunoregulatory cells for the organism to resist infection and prevent malignant transformation of cells, and play a very important role in immune surveillance and immune defense. The past studies show that the proportion, number and function of the NK cells may be reduced in the development of certain diseases, such as tumors, autoimmune diseases, aging-related diseases and HIV infection or the like. The US Disease Control Center reported in 2012 that the vitality and number of NK cells are important factors for ensuring human body health. The onsets of almost all diseases are related to apparent insufficiency of vitality of the NK cells. When people have a disease, either chronic or sporadic or acute, the vitality of the NK cells is below an average level. A good disease treatment is to recover the NK cells to a high vitality level. Different from the T lymphocytes, the NK cells have the following characteristics. The NK cells can directly lyse and destroy tumor cells and virus-infected cells without pre-stimulation. Cell membranes are degraded by secreting perforin, serine proteases such as granzymes A and B, and molecules such as chondroitin sulfate proteoglycans, so as to destroy the integrity of target cells and achieve a cytolytic effect. Apoptosis of the target cells may be caused when NK cell surface expression Fas ligand (FasL) and TNF-related apoptosis-inducing ligand (TRAIL) bind with receptors on the target cells. Activated NK cells will release a large number of cytokines such as interferon-γ and tumor necrosis factor-α. The NK cells bind with an Fc segment of an antibody via cell membrane FcγRIII (CD16) to mediate antibody-dependent cytotoxicity. With the understanding of the biological function and activation mechanism of the NK cells, an immunotherapy using the NK cells as target points is of importance to an early prevention, control and treatment of diseases.

In recent years, immunotherapy has become the fourth largest treatment mode after surgery, radiotherapy-chemotherapy and endocrine therapy, and has gradually received more and more attentions. Adoptive cellular immunotherapy, as one of the immunotherapy methods, has received major developments in clinical studies. Currently, clinical studies have been carried out, in which autologous NK cells are applied to hematological tumors (acute myeloid leukemia, acute lymphocytic leukemia, etc.) and solid tumors (glioma, renal cancer and malignant melanoma) by adoptive immunotherapy. The clinical studies are confirmed to be safe, but the results of clinical trials are not so satisfactory. Specifically, by reinfusing autologous peripheral blood after an NK cell sorting is performed on the autologous peripheral blood, the survival rate and recurrence rate of patients are not improved as compared with a control group, although it is observed that the killing capability of the NK cells on tumor cells is improved, which may be related to an immune evasion prone to being formed for the autologous NK cells during the development of the tumor. In addition, subjects for the clinical trials of the NK cell adoptive immunotherapy are often extremely advanced patients who have experienced various chemotherapy failures, have a heavy tumor burden, a poor organism immunity, and NK cells with serious deficient functions.

To improve the efficacy of clinical treatment of the NK cells, it is necessary to change the previous treatment strategy. In order to overcome these defects, NK cells of an allogeneic origin are good choice. It is discovered by some scholars via research that the recurrence of leukemia can be delayed without causing graft-versus-host disease if leukemia patients are treated by transplanting allogeneic NK cells, and this has drawn people's attention to allogeneic reactivity of NK cells. It can be seen from the results of current clinical studies, heterologous NK cells are significantly superior to autologous NK cells in terms of complete remission rate, event-free survival, recurrence rate, and mortality. Furthermore, the heterologous NK cells are not accompanied by graft-versus-host reaction. The killing ability of NK cells to tumor cells depends on a regulation of signal balance between NK cell surface activating receptors and NK cell surface inhibitory receptors. Killer cell immunoglobulin-like receptors (KIR) form a receptor family which binds with major histocompatibility class I (MHC-I), and play an important role in regulating the activation threshold of human NK cells. Recently, it is shown by more and more studies that the killing ability of the NK cells to receptor tumor cells and the effect of NK cell adoptive immunotherapy both can be greatly improved by screening an optimum donor when using the mismatch of KIR receptor/MHC-I ligand. Currently, the KIR-based donor selection has received great attention in improving hematopoietic stem cell/bone marrow transplantation and adoptive immunotherapy for tumors. The heterologous NK infusion has become a new strategy for clinical NK cell adoptive immunotherapy.

Cord blood, as a ready-made heterologous resource, has no ethical problem and has rich sources, and it is easy to find the optimum donor of the cord blood. In addition, T lymphocytes in the cord blood develop immaturely and most of them are juvenile cells. Hence, after a transplantation, acute and chronic graft-versus-host diseases have a low probability of occurrence and a light degree of severity, so the cord blood has become a very promising source of heterologous NK cells. For a clinical infusion of NK cells, a sufficient number of cells with a sufficient purity are required. However, the NK cells account for a small proportion of cord blood cells, and the number of the NK cells is small. For clinical application, a large quantity of high-purity NK cells have to be obtained via ex vivo separation and culturing expansion. Therefore, how to obtain high-purity, high-quality NK cells has become the key issue of the NK cell adoptive immunotherapy (Document 1: Klingemann H: Challenges of cancer therapy with natural killer cells, Cytotherapy 2015, 17(3): 245-249.).

At present, there are mainly two methods for separating and expanding cord blood NK cells (as shown in FIG. 1). In method 1, the NK cells account for about 10% to 20% of lymphocytes in the cord blood, have immature functions and have a poor ability of killing tumor cells. The NK cells can be sorted and enriched by using a flow cytometer or a magnetic bead sorter. Then, the number of NK cells is increased and the ability of the NK cells to kill tumors is improved by ex vivo expansion. The Celgene Cellular Therapeutics Company separates mononuclear cells from frozen resuscitation cord blood by using lymphocyte separation solution, and sorts CD56⁺CD3⁻NK cells by using the EasySep®NK enrichment kit (StemCell Technologies, the kit containing CD3, CD4, CD14, CD19, CD20, CD36, CD66b, CD123, HLA-DR and glycophorin A antibodies); the purity of the sorted cells can be up to 71%, and the number of the cells per part of cord blood is 1.5×10⁷. Then, trophoblast cells (mitomycin C-treated peripheral blood mononuclear cells and K562 cells) are added via a starting culturing medium (including: Iscoves modified Dulbecco medium (IMDM), 10% fatal bovine serun (FBS), 35 mg/mL transferrin, 5 μg/mL insulin, 20 μM ethanolamine, 1 μg/mL unsaturated fatty acid, 1 μg/mL linoleic acid, 0.2 μg/mL palmitic acid, 2.5 μg/mL bovine serum albumin, 0.1 μg/mL plant lectin, 1% cyan-streptomycin and 200 IU/mL interleukin-2). After being cultured under a condition of 37° C. and 5% CO₂ for 5-7 days, the NK cells are transferred to a maintenance culturing medium (IMDM, 10% FBS, 2% human AB serum, 1% cyan-streptomycin, 200 IU/mL interleukin-2) and cultured for 21 days. Then, CD56⁺CD3⁻NK cells may be obtained for each part of cord blood, with a cell purity greater than 80% and an average total number of cells being 1.2×10⁹ per part of cord blood (Document 2: Kang L, Voskinarian-Berse V, Law E, Reddin T, Bhatia M, Hariri A, Ning Y, Dong D, Maguire T, Yarmush M et al: Characterization and ex vivo Expansion of Human Placenta-Derived Natural Killer Cells for Cancer Immunotherapy. Frontiers in immunology 2013, 4:101.). In method 2, NK cells start from hematopoietic stem cells. The cord blood is rich in hematopoietic stem cells. The hematopoietic stem cells are sorted and enriched by using a flow cytometer or a magnetic bead sorter, and a certain number of mature NK cells are obtained by ex vivo differentiation. The Glycostem Therapeutics Company sorts and enriches CD34⁺ hematopoietic stem cells with a purity of 67% and the number of 3.8×10⁶ per part of cord blood, from frozen resuscitation cord blood by using a CliniMACS CD34 magnetic bead sorting kit. The cells are cultured for 9 days in cell expansion culturing medium I (including: GBGM® culturing medium, 10% human serum, high-dose cytokine combination (SCF, Flt3L, TP0, IL-7, low-molecular-weight heparin) and low-dose factor combination (GM-CSF, G-CSF, IL-6). Then, the cells are expanded for 14 days in cell expansion culturing medium II (TP0 is replaced with IL-15, and other components are the same as the culturing medium I), and finally, the cells are differentiated and cultured for 35 days in an NK cell differentiation culturing medium (low-dose factor combination is replaced with (IL-7, SCF, IL-15 and IL-2), and others are the same as the culturing medium I), to obtain NK cells with a purity greater than 90% and an average number of NK cells of 2×10⁹ per part of the cord blood (Document 3: Spanholtz J, Preijers F, Tordoir M, Trilsbeek C, Paardekooper J, de Witte T, Schaap N, Dolstra H: Clinical-grade generation of active NK cells from cord blood hematopoietic progenitor cells for immunotherapy using a closed-system culture process, PloS one 2011, 6 (6): e20740.).

In summary, the methods of preparing cord blood NK cells in the prior art have the following disadvantages.

1: cells (NK cells or hematopoietic stem cells) have to be sorted, the sorting technology affects the activity of the NK cells, and a sorter is also required.

2: trophoblast cells are required, and an introduction of K562 cells greatly affects the safety of expanding the NK cells.

3: the operation is complicated and the technical versatility is poor.

4: the costs for separation stage and expansion stage are high.

5: the safety is poor due to the use of animal-source composition additives.

6: the yield of NK cells is low.

### SUMMARY

A first object of the present disclosure is to provide a simple and safe ex vivo expansion method for cord blood NK cells.

The ex vivo expansion method for cord blood NK cells according to the present disclosure includes:

step 1): activatedly culturing the cord blood NK cells by: adjusting the density of cord blood mononuclear cells to 0.5×10⁶ to 5×10⁶/mL using a lymphocyte culturing medium, adding at least zoledronic acid with the concentration ranging from 1 μg/mL to 10 μg/mL and recombinant human interleukin-2 with the concentration ranging from 200 IU/mL to 2000 IU/mL, culturing for 1 to 5 days in an environment with a temperature ranging from 36° C. to 40° C. and CO₂ saturated humidity of 5%, and collecting cell fluid; and step 2): proliferatedly culturing the cord blood NK cells by: obtaining cells from the cell fluid collected in step 1), adjusting the density of the cells to 0.5×10⁶ to 5×10⁶/mL using a lymphocyte culturing medium, adding recombinant human interleukin-2 with the concentration ranging from 200 IU/mL to 2000 IU/mL, culturing in an environment with a temperature ranging from 36° C. to 38° C. and CO₂ saturated humidity of 5%, replenishing with fresh culturing medium every other 2 to 3 days and adjusting the density of the cells to 0.5×10⁶ to 5×10⁶/mL, and culturing for 14 to 35 days to harvest the cord blood NK cells.

The cord blood mononuclear cells used in step 1) are separated from fresh anticoagulation cord blood or frozen resuscitation cord blood by:

1.1: taking the fresh anticoagulation cord blood or frozen resuscitation cord blood, and diluting it with PBS of a volume which is 1 to 2 times the volume of the cord blood;

1.2: adding slowly the diluted cord blood from above to the lymphocyte separation solution, and maintaining a clear interface between the diluted cord blood and the lymphocyte separation solution, wherein the volume of the diluted cord blood is equal to the volume of the lymphocyte separation solution;

1.3: centrifuging for 20 to 30 minutes at a room temperature and with a centrifugal force of 980 g; and

1.4: after the centrifugation, aspirating a white membrane-like mononuclear cell layer, which is a second layer from the top, and washing the mononuclear cell layer with the PBS, to obtain cord blood mononuclear cells.

The lymphocyte culturing medium used in step 1) may be AIM V® Medium CTS™ (commercially available from Life Technology Company, USA) or GMP S&XFM™-CD lymphocyte culturing medium (the same name product of Beijing Jing-Meng Stem Cell Technology Co., Ltd.), and is preferably the GMP S&XFM™-CD lymphocyte culturing medium.

In the case that the lymphocyte culturing medium used in step 1) is the AIM V® Medium CTS™, the activated culturing preferably is: adjusting the density of the cells to 1×10⁶ to 3×10⁶/mL using the AIM V® Medium CTS™ lymphocyte culturing medium, adding zoledronic acid with the concentration ranging from 1 μg/mL to 5 μg/mL and recombinant human interleukin-2 with the concentration ranging from 500 IU/mL to 1500 IU/mL, and culturing for 2 to 4 days in an environment with a temperature ranging from 36° C. to 38° C. and CO₂ saturated humidity of 5%; the activatedly culturing more preferably is: adjusting the density of the cells to 2×10⁶/mL using the AIM V® Medium CTS™ lymphocyte culturing medium, adding zoledronic acid with the concentration of 2 μg/mL and recombinant human interleukin-2 with the concentration of 1000 IU/mL, and culturing for 3 days in an environment with a temperature of 37° C. and CO₂ saturated humidity of 5%.

In the case that the lymphocyte culturing medium used in step 1) is the GMP S&XFM™-CD, the activatedly culturing preferably is one of the followings:

A: adjusting the density of the cells to 1×10⁶ to 3×10⁶/mL using the GMP S&XFM™-CD lymphocyte culturing medium, adding zoledronic acid with the concentration ranging from 1 μg/mL to 5 μg/mL and recombinant human interleukin-2 with the concentration ranging from 500 IU/mL to 1500 IU/mL, and culturing for 2 to 4 days in an environment with a temperature ranging from 36° C. to 38° C. and CO₂ saturated humidity of 5%; and the activatedly culturing more preferably is: adjusting the density of the cells to 2×10⁶/mL using the GMP S&XFM™-CD lymphocyte culturing medium, adding zoledronic acid with the concentration of 2 μg/mL and recombinant human interleukin-2 with the concentration of 1000 IU/mL, and culturing for 3 days in an environment with a temperature of 37° C. and CO₂ saturated humidity of 5%;

B: adjusting the density of the cells to 1×10⁶ to 3×10⁶/mL using the GMP S&XFM™-CD lymphocyte culturing medium, adding zoledronic acid with the concentration ranging from 1 μg/mL to 5 μg/mL, recombinant human interleukin-2 with the concentration ranging from 500 IU/mL to 1500 IU/mL, recombinant human interleukin-15 with the concentration ranging from 1 ng/mL to 100 ng/mL (preferably from 1 ng/mL to 20 ng/mL) and recombinant human interleukin-18 with the concentration ranging from 1 ng/mL to 100 ng/mL (preferably from 1 ng/mL to 20 ng/mL), and culturing for 2 to 4 days in an environment with a temperature ranging from 36° C. to 38° C. and CO₂ saturated humidity of 5%; the activatedly culturing more preferably is: adjusting the density of the cells to 2×10⁶/mL using the GMP S&XFM™-CD lymphocyte culturing medium, adding zoledronic acid with the concentration of 2 μg/mL, recombinant human interleukin-2 with the concentration of 1000 IU/mL, recombinant human interleukin-15 with the concentration of 10 ng/mL and recombinant human interleukin-18 with the concentration of 10 ng/mL, and culturing for 3 days in an environment with a temperature of 37° C. and CO₂ saturated humidity of 5%;

C: adjusting the density of the cells to 1×106 to 3×106/mL using the GMP S&XFM™-CD lymphocyte culturing medium, adding zoledronic acid with the concentration ranging from 1 μg/mL to 5 μg/mL, recombinant human interleukin-2 with the concentration ranging from 500 IU/mL to 1500 IU/mL, recombinant human interleukin-15 with the concentration ranging from 1 ng/mL to 100 ng/mL (preferably from 1 ng/mL to 20 ng/mL) and recombinant human interleukin-18 with the concentration ranging from 1 ng/mL to 100 ng/mL (preferably from 1 ng/mL to 20 ng/mL), and culturing for 0.5 to 1 day in an environment with a temperature ranging from 38.5° C. to 39.5° C. and CO₂ saturated humidity of 5%; the activatedly culturing more preferably is: adjusting the density of the cells to 2×106/mL using the GMP S&XFM™-CD lymphocyte culturing medium, adding zoledronic acid with the concentration of 2 μg/mL, recombinant human interleukin-2 with the concentration of 1000 IU/mL, recombinant human interleukin-15 with the concentration of 10 ng/mL and recombinant human interleukin-18 with the concentration of 10 ng/mL, and culturing for 1 day in an environment with a temperature of 39° C. and CO₂ saturated humidity of 5%.

The GMP S&XFM™-CD lymphocyte separation solution (the same name product of Beijing Jing-Meng Stem Cell Technology Co., Ltd., the same as sample density separation solution of medical device with record No.: Jinghai Machinery Equipment No. 20150002) is preferably used in proliferated culturing cord blood NK cells in step 2), and the operation preferably is: pipetting the cell fluid to a centrifuge tube, performing a centrifugation for 10 minutes with a centrifugal force of 150 g, discarding the supernatant, adjusting the density of the cells to 0.5×10⁶ to 2×10⁶/mL (more preferably 1×10⁶/mL) using the GMP S&XFM™-CD lymphocyte culturing medium, adding recombinant human interleukin-2 with the concentration ranging from 500 IU/mL to 1500 IU/mL (more preferably 1000 IU/mL), culturing in an environment with a temperature of 37° C. and CO₂ saturated humidity of 5%, replenishing with fresh medium every other 2 to 3 days and adjusting the density of the cells to 0.5×10⁶ to 2×10⁶/mL (more preferably 1×10⁶/mL), adding recombinant human interleukin-2 with the concentration ranging from 500 IU/mL to 1500 IU/mL (more preferably 1000 IU/mL), and culturing for 18 to 24 days (more preferably 21 days), to obtain the cord blood NK cells.

A dedicated activation culturing medium used in step 1) of the above ex vivo expansion method for the cord blood NK cells also belongs to the contents of the present disclosure. The dedicated activation culturing medium is one of the following formulations:

an AIM V® Medium CTS™ lymphocyte culturing medium added with zoledronic acid with the concentration ranging from 1 μg/mL to 10 μg/mL and recombinant human interleukin-2 with the concentration ranging from 200 IU/mL to 2000 IU/mL;

preferably, an AIM V® Medium CTS™ lymphocyte culturing medium added with zoledronic acid with the concentration ranging from 1 μg/mL to 5 μg/mL and recombinant human interleukin-2 with the concentration ranging from 500 IU/mL to 1500 IU/mL; and more preferably, an AIM V® Medium CTS™ lymphocyte culturing medium added with zoledronic acid with the concentration of 2 μg/mL and recombinant human interleukin-2 with the concentration of 1000 IU/mL;

a GMP S&XFM™-CD lymphocyte culturing medium added with zoledronic acid with the concentration ranging from 1 μg/mL to 10 μg/mL and recombinant human interleukin-2 with the concentration ranging from 200 IU/mL to 2000 IU/mL;

preferably, a GMP S&XFM™-CD lymphocyte culturing medium added with zoledronic acid with the concentration ranging from 1 μg/mL to 5 μg/mL and recombinant human interleukin-2 with the concentration ranging from 500 IU/mL to 1500 IU/mL; and

more preferably, a GMP S&XFM™-CD lymphocyte culturing medium added with zoledronic acid with the concentration of 2 μg/mL and recombinant human interleukin-2 with the concentration of 1000 IU/mL; or

a GMP S&XFM™-CD lymphocyte culturing medium added with zoledronic acid with the concentration ranging from 1 μg/mL to 10 μg/mL, recombinant human interleukin-2 with the concentration ranging from 200 IU/mL to 2000 IU/mL, recombinant human interleukin-15 with the concentration ranging from 1 ng/mL to 100 ng/mL and recombinant human interleukin-18 with the concentration ranging from 1 ng/mL to 100 ng/mL;

preferably, a GMP S&XFM™-CD lymphocyte culturing medium added with zoledronic acid with the concentration ranging from 1 μg/mL to 5 μg/mL, recombinant human interleukin-2 with the concentration ranging from 500 IU/mL to 1500 IU/mL, recombinant human interleukin-15 with the concentration ranging from 1 ng/mL to 20 ng/mL and recombinant human interleukin-18 with the concentration ranging from 1 ng/mL to 20 ng/mL; and

more preferably, a GMP S&XFM™-CD lymphocyte culturing medium added with zoledronic acid with the concentration of 2 μg/mL, recombinant human interleukin-2 with the concentration of 1000 IU/mL, recombinant human interleukin-15 with the concentration of 10 ng/mL and recombinant human interleukin-18 with the concentration of 10 ng/mL.

A dedicated proliferation culturing medium used in step 2) of the above ex vivo expansion method for the cord blood NK cells also belongs to the content of the present disclosure. The dedicated proliferation culturing medium is a GMP S&XFM™-CD lymphocyte culturing medium added with recombinant human interleukin-2 with the concentration ranging from 200 IU/mL to 2000 IU/mL (preferably 1000 IU/mL).

The cord blood NK cells obtained by the above ex vivo expansion method also belong to the content of the present disclosure, and the purity of the obtained cells exceeds 90%.

An application of the cord blood NK cells obtained by using the method according to the present disclosure in preparing tumor immunotherapy drugs or in a tumor immunotherapy also belongs to the content of the present disclosure.

Another object of the present disclosure is to provide an ex vivo expansion kit for cord blood NK cells.

The ex vivo expansion kit for the cord blood NK cells according to the present disclosure mainly includes the following reagents:

1): lymphocyte separation solution of the cord blood NK cells (the same name product of Beijing Jing-Meng Stem Cell Technology Co., Ltd.), or other commercially available lymphocyte separation solutions;

2): a cord blood NK cell activation culturing medium, including: an AIM V® Medium CTS™ culturing medium (Life Technology, USA) or a GMP S&XFM™-CD lymphocyte culturing medium (product of Beijing Jing-Meng Stem Cell Technology Co., Ltd.), and zoledronic acid, recombinant human interleukin-2, recombinant human interleukin-15 and recombinant human interleukin-18; and

3): a cord blood NK cell proliferation culturing medium, including: a GMP S&XFM™-CD lymphocyte culturing medium (product of Beijing Jing-Meng Stem Cell Technology Co., Ltd.) and recombinant human interleukin-2.

The reagents in the ex vivo expansion kit for the cord blood NK cells is used based on the above ex vivo expansion method for the cord blood NK cells.

With the above solutions, a simply operable and safe ex vivo expansion method and relevant kit for the cord blood NK cells are provided according to the present disclosure. The present disclosure has the following advantageous effects as compared with existing methods for separating and expanding cord blood NK cells.

1): Cord blood mononuclear cells are directly used and no cell sorting step is required. That is, it is unnecessary to sort NK cells or hematopoietic stem cells, thereby simplifying a cell preparing process and greatly reducing preparing cost of the cord blood NK cells.

2): No trophoblast cells are required, and a safety risk brought by the trophoblast cells to the prepared cord blood NK cells is avoided.

3): No animal-source compositions are contained in the cell culturing medium used for preparing the NK cells, hence the safety is good. In addition, the chemical compositions are clear so that the stability between batches is improved.

4): The yield of NK cells is high, the purity of the obtained cord blood NK cells is above 90%, and the total number of cells can reach 10¹⁰⁻¹¹ per part of cord blood.

The ex vivo expansion method and kit for cord blood NK cells according to the present disclosure both play important roles in preparing tumor immunotherapy drugs and in tumor immunotherapy, and have broad application prospects.

### BRIEF DESCRIPTION OF THE DRAWINGS

FIG. 1 shows an existing method for separating and expanding cord blood NK cells;

FIG. 2 shows a workflow of an ex vivo expansion method for cord blood NK cells according to the present disclosure;

FIG. 3 is a diagram showing cell morphologies of fresh cord blood NK cells and frozen resuscitation cord blood NK cells before and after an ex vivo expansion;

FIG. 4 is a diagram showing a growth curve of ex vivo expansion of fresh cord blood NK cells and a growth curve of ex vivo expansion of frozen resuscitation cord blood NK cells;

FIG. 5 shows results of the purities of the fresh cord blood NK cells and frozen resuscitation cord blood NK cells before and after ex vivo expansion, detected by a flow cytometer; and

FIG. 6 shows the tumor target cell killing effect of ex vivo expanded fresh cord blood NK cells and the tumor target cell killing effect of ex vivo expanded frozen resuscitation cord blood NK cells.

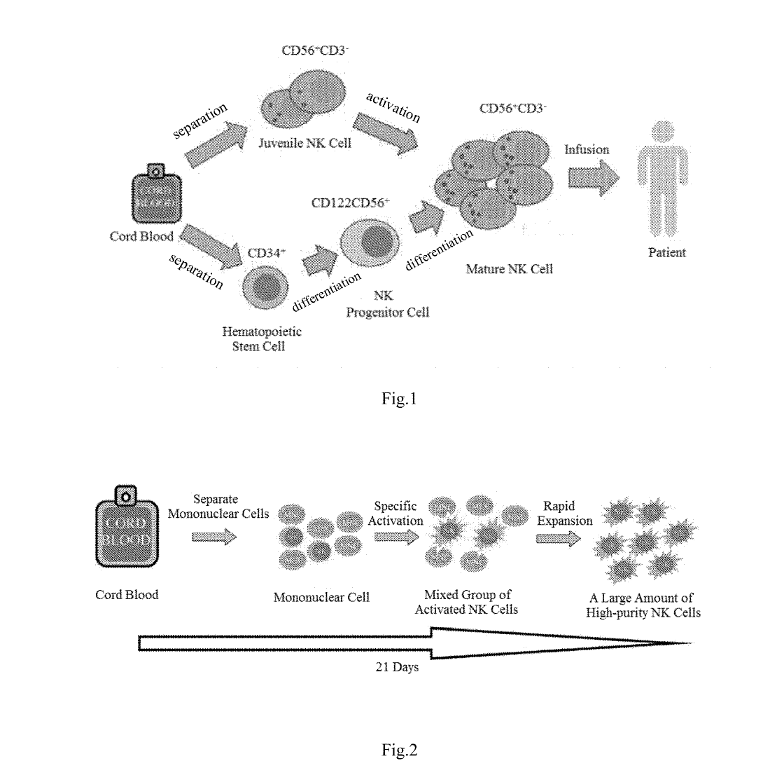

### DETAILED DESCRIPTION

In view of the deficiency in separating and expanding cord blood NK cells in the prior art, the present disclosure aims to provide a method for ex vivo preparing cord blood NK cells, and a dedicated activation culturing medium and a dedicated proliferation culturing medium which are used in the method.

An ex vivo expansion method for cord blood NK cells may include the following steps.

In step 1), the cord blood NK cells are activatedly cultured by: adjusting the density of cord blood mononuclear cells to 0.5×10⁶ to 5×10⁶/mL (preferably from 1×10⁶ to 3×10⁶/mL, and most preferably 2×10⁶/mL) using a lymphocyte culturing medium (described in detail later), inoculating cell suspension in a cell culture flask, adding zoledronic acid with the concentration ranging from 1 μg/mL to 10 μg/mL (preferably 1 μg/mL to 5 μg/mL, and most preferably 2 μg/mL) and recombinant human interleukin-2 (for forming a dedicated activation culturing medium) with the concentration ranging from 200 IU/mL to 2000 IU/mL (preferably 500 IU/mL to 1500 IU/mL, and most preferably 1000 IU/mL), and culturing for 1 to 5 days in an environment with a temperature ranging from 36° C. to 40° C. and CO₂ saturated humidity of 5%.

Specifically, the lymphocyte culturing medium used in step 1) may be AIM V® Medium CTS™ (purchased from Life Technology, USA) or GMP S&XFM™-CD lymphocyte culturing medium (the same name product of Beijing Jing-Meng Stem Cell Technology Co., Ltd., the same as the cell culture medium of medical device with record No.: Jinghai Machinery Equipment No. 20150008), and is preferably the GMP S&XFM™-CD lymphocyte culturing medium. For detailed description of the GMP S&XFM™-CD lymphocyte culturing medium, reference may also be made to Document 4 CN103146648A with patent application No. 201310082166.8.

Different dedicated activation culturing medium are correspondingly obtained according to the different types of lymphocyte culturing medium as selected.

a) In the case that AIM V® Medium CTS™ lymphocyte culturing medium is selected, the composition of the dedicated activation culturing medium is: the AIM V® Medium CTS™ lymphocyte culturing medium added with zoledronic acid with the concentration ranging from 1 μg/mL to 10 μg/mL (preferably from 1 μg/mL to 5 μg/mL, and most preferably 2 μg/mL) and recombinant human interleukin-2 with the concentration ranging from 200 IU/mL to 2000 IU/mL (preferably from 500 IU/mL to 1500 IU/mL, and most preferably 1000 IU/mL).

With the dedicated activation culturing medium, the activatedly culturing method in step 1) preferably is: adjusting the density of cord blood mononuclear cells to 0.5×10⁶ to 5×10⁶/mL (preferably from 1×10⁶ to 3×10⁶/mL, and most preferably 2×10⁶/mL) using the AIM V® Medium CTS™ lymphocyte culturing medium, inoculating cell suspension in a cell culture flask, adding zoledronic acid with the concentration ranging from 1 μg/mL to 10 μg/mL (preferably from 1 μg/mL to 5 μg/mL, and most preferably 2 μg/mL) and recombinant human interleukin-2 with the concentration ranging from 200 IU/mL to 2000 IU/mL (preferably from 500 IU/mL to 1500 IU/mL, and most preferably 1000 IU/mL) into the AIM V® Medium CTS™ lymphocyte culturing medium, and culturing in an environment with a temperature ranging from 36° C. to 38° C. (preferably 37° C.) and CO₂ saturated humidity of 5% for 1 to 5 days (preferably 2 to 4 days, most preferably 3 days).

b) In the case that the GMP S&XFM™-CD lymphocyte culturing medium is selected, the composition of the dedicated activation culturing medium is: the GMP S&XFM™-CD lymphocyte culturing medium added with zoledronic acid with the concentration ranging from 1 μg/mL to 10 μg/mL (preferably from 1 μg/mL to 5 μg/mL, and most preferably 2 μg/mL) and recombinant human interleukin-2 with the concentration ranging from 200 IU/mL to 2000 IU/mL (preferably from 500 IU/mL to 1500 IU/mL, and most preferably 1000 IU/mL).

With the dedicated activation culturing medium, the activatedly culturing method in step 1) preferably is: adjusting the density of cord blood mononuclear cells to 0.5×10⁶ to 5×10⁶/mL (preferably from 1×10⁶ to 3×10⁶/mL, and most preferably 2×10⁶/mL) using the GMP S&XFM™-CD lymphocyte culturing medium, inoculating cell suspension in a cell culture flask, adding zoledronic acid with the concentration ranging from 1 μg/mL to 10 μg/mL (preferably from 1 μg/mL to 5 μg/mL, and most preferably 2 μg/mL) and recombinant human interleukin-2 with the concentration ranging from 200 IU/mL to 2000 IU/mL (preferably from 500 IU/mL to 1500 IU/mL, and most preferably 1000 IU/mL) into the GMP S&XFM™-CD lymphocyte culturing medium, and culturing in an environment with a temperature ranging from 36° C. to 38° C. (preferably 37° C.) and CO₂ saturated humidity of 5% for 1 to 5 days (preferably 2 to 4 days, most preferably 3 days).

c) In the case that the GMP S&XFM™-CD lymphocyte culturing medium is selected, the composition of the dedicated activation culturing medium is: the GMP S&XFM™-CD lymphocyte culturing medium added with zoledronic acid with the concentration ranging from 1 μg/mL to 10 μg/mL (preferably from 1 μg/mL to 5 μg/mL, and most preferably 2 μg/mL), recombinant human interleukin-2 with the concentration ranging from 200 IU/mL to 2000 IU/mL (preferably from 500 IU/mL to 1500 IU/mL, and most preferably 1000 IU/mL), recombinant human interleukin-15 with the concentration ranging from 1 ng/mL to 100 ng/mL (preferably from 1 ng/mL to 20 ng/mL, and most preferably 10 ng/mL) and recombinant human interleukin-18 with the concentration ranging from 1 ng/mL to 100 ng/mL (preferably from 1 ng/mL to 20 ng/mL, and most preferably 10 ng/mL).

With the dedicated activation culturing medium, the activatedly culturing method in step 1) preferably is: adjusting the density of cord blood mononuclear cells to 0.5×10⁶ to 5×10⁶/mL (preferably from 1×10⁶ to 3×10⁶/mL, and most preferably 2×10⁶/mL) using the GMP S&XFM™-CD lymphocyte culturing medium, inoculating cell suspension into a cell culture flask, adding zoledronic acid with the concentration ranging from 1 μg/mL to 10 μg/mL (preferably from 1 μg/mL to 5 μg/mL, and most preferably 2 μg/mL), recombinant human interleukin-2 with the concentration ranging from 200 IU/mL to 2000 IU/mL (preferably from 500 IU/mL to 1500 IU/mL, and most preferably 1000 IU/mL), recombinant human interleukin-15 with the concentration ranging from 1 ng/mL to 100 ng/mL (preferably from 1 ng/mL to 20 ng/mL, and most preferably 10 ng/mL) and recombinant human interleukin-18 with the concentration ranging from 1 ng/mL to 100 ng/mL (preferably from 1 ng/mL to 20 ng/mL, and most preferably 10 ng/mL) into the GMP S&XFM™-CD lymphocyte culturing medium, and culturing in an environment with a temperature ranging from 36° C. to 38° C. (preferably 37° C.) and CO₂ saturated humidity of 5% for 1 to 5 days (preferably 2 to 4 days, most preferably 3 days).

By using the dedicated activation culturing medium and changing the culturing conditions, for example, culturing in an environment with a temperature ranging from 38° C. to 40° C. (preferably from 38.5° C. to 39.5° C., and most preferably 39° C.) and CO₂ saturated humidity of 5% for 0.5 to 3 days (preferably 0.5 to 1 day, most preferably 1 day), optimum conditions for activatedly culturing the cord blood NK cells in step 1) are obtained.

In step 2), the cord blood NK cells are proliferatedly cultured by: pipetting the cell fluid obtained in step 1) to a centrifuge tube, performing a centrifugation for 10 minutes with a centrifugal force of 150 g, discarding the supernatant, adjusting the density of the cells to 0.5×10⁶ to 5×10⁶/mL (preferably from 0.5×10⁶ to 2×10⁶/mL, and most preferably 1×10⁶/mL) using the GMP S&XFM™-CD lymphocyte culturing medium, inoculating cell suspension into a cell culture flask, adding recombinant human interleukin-2 (for forming the dedicated proliferation culturing medium) with the concentration ranging from 200 IU/mL to 2000 IU/mL (preferably from 500 IU/mL to 1500 IU/mL, and most preferably 1000 IU/mL), culturing in an environment with a temperature ranging from 36° C. to 38° C. (preferably 37° C.) and CO₂ saturated humidity of 5%, replenishing with fresh GMP S&XFM™-CD lymphocyte culturing medium every other 2 to 3 days and adjusting the density of the cells to 0.5×10⁶/mL to 5×10⁶/mL (preferably from 0.5×10⁶/mL to 2×10⁶/mL, and most preferably 1×10⁶/mL), adding recombinant human interleukin-2 with the concentration ranging from 200 IU/mL to 2000 IU/mL (preferably from 500 IU/mL to 1500 IU/mL, and most preferably 1000 IU/mL), and culturing for 14 to 35 days (preferably 18 to 24 days, and most preferably 21 days), to obtain the cord blood NK cells.

The composition of the dedicated proliferation culturing medium in the proliferatedly culturing in step 2) is: GMP S&XFM™-CD lymphocyte culturing medium added with recombinant human interleukin-2 with the concentration ranging from 200 IU/mL to 2000 IU/mL (preferably from 500 IU/mL to 1500 IU/mL, and most preferably 1000 IU/mL).

The proliferatedly culturing of the cord blood NK cells in step 2) preferably is: pipetting the cell fluid to a centrifuge tube, centrifuging the cell fluid for 10 minutes with a centrifugal force of 150 g (unit of the centrifugal force), discarding the supernatant, adjusting the density of the cells to 1×10⁶/mL using the GMP S&XFM™-CD lymphocyte culturing medium, inoculating cell suspension into a cell culture flask, adding recombinant human interleukin-2 with the concentration of 1000 IU/mL into the cell culture flask, culturing in an environment with a temperature of 37° C. and CO₂ saturated humidity of 5% for 21 days, during which fresh culturing medium is replenished with every other 2 to 3 days and the density of cells is adjusted to 1×10⁶/mL, to obtain the cord blood NK cells.

In the above method, the cord blood mononuclear cells used in step 1) are separated from the cord blood by the following steps 1.1 to 1.4.

In step 1.1, 100 mL of fresh anticoagulation cord blood or frozen resuscitation cord blood is taken, and 100 mL of PBS is added to dilute the cord blood.

In step 1.2, 20 mL of lymphocyte separation solution (the same name product of Beijing Jing-Meng Stem Cell Technology Co., Ltd., the same as sample density separation solution of medical device with record No.: Jinghai Machinery Equipment No. 20150002; for details of the separation solution, reference may be made to Document 5 CN102533650A with patent No. ZL 201110456878.2,) is respectively added to ten centrifuge tubes each of a volume of 50 mL. The 200 mL of the diluted cord blood is slowly and uniformly added from above to the separation solution surface respectively, that is, 20 mL of diluted cord blood is added to each of the centrifuge tubes to maintain a clear interface between the two liquids. The separation solution used in this step may be other commercially available lymphocyte separation solutions.

In step 1.3, centrifugation is performed for 20 to 40 minutes (preferably 30 minutes) at a room temperature and with a centrifugal force ranging from 400 g to 1200 g (preferably 980 g, where g is a unit of centrifugal force. If other commercially available lymphocyte separation solutions are used in step 1.2, then an operation is executed according to the instructions of the other commercially available lymphocyte separation solutions).

In step 1.4, after the centrifugation, the solution in the centrifuge tube is divided into four layers with clear interfaces from top to bottom. The four layers are sequentially a light yellow plasma layer, a white membrane-like mononuclear cell layer, a transparent separation solution layer and a red erythrocyte layer from top to bottom. The white membrane-like mononuclear cell layer is carefully aspirated and put into another centrifuge tube, and is washed with PBS to obtain cord blood mononuclear cells.

Based on the above method, an ex vivo expansion kit for cord blood NK cells is further provided according to the present disclosure, which mainly includes the following reagents:

1): lymphocyte separation solution of the cord blood NK cells (the same name product of Beijing Jing-Meng Stem Cell Technology Co., Ltd., the same as sample density separation solution of medical device with record No.: Jinghai Machinery Equipment No. 20150002), or other commercially available lymphocyte separation solutions;

2): a cord blood NK cell activation culturing medium, including: an AIM V® Medium CTS™ medium (purchased from Life Technology, USA) or a GMP S&XFM™-CD lymphocyte culturing medium (the same name product of Beijing Jing-Meng Stem Cell Technology Co., Ltd., the same as cell culture medium of medical device with record No.: Jinghai Machinery Equipment No. 20150008), and other reagents described in the above steps 1) to 4), including zoledronic acid, recombinant human interleukin-2, recombinant human interleukin-15 and recombinant human interleukin-18; and

3): a cord blood NK cell proliferation culturing medium, including: a GMP S&XFM™-CD lymphocyte culturing medium (the same name product of Beijing Jing-Meng Stem Cell Technology Co., Ltd., the same as cell culture medium of medical device with record No.: Jinghai Machinery Equipment No. 20150008) and the above described reagent: recombinant human interleukin-2.

Hereinafter, the present disclosure is further described in conjunction with embodiments, and methods used in the embodiments are all conventional methods, unless otherwise stated.

Unless otherwise stated, the percentage concentration is a mass/mass (W/W, unit: g/100 g) percentage concentration, mass/volume (W/V, unit: g/100 mL) percentage concentration or volume/volume (V/V, unit: mL/100 mL) percentage concentration.

The approaches for obtaining various biomaterials described in the embodiments are only intended to provide an experimental means for carrying out the present disclosure, and should not be considered as limiting the sources of the biomaterials of the present disclosure. In fact, the sources of the adopted biomaterials are extensive, and any biomaterial that can be obtained without violating laws and ethics can be used for replacement according to the instructions in the embodiments.

The embodiments are implemented according to the technical solutions of the present disclosure, and detailed embodiments and specific operation processes are given. The embodiments are helpful for understanding the present disclosure, but the scope of protection of the present disclosure is not limited to the following embodiments.

First embodiment: ex vivo expansion and detection of cord blood NK cells

I: ex vivo expansion of the cord blood NK cells (the first activatedly culturing scheme)

As shown in FIG. 1, the ex vivo expansion method for the cord blood NK cells according to the present disclosure includes the following steps.

In step 1), cord blood mononuclear cells are separated. Specifically:

In sub-step 1.1, 100 mL of fresh anticoagulation cord blood (sample 1) and 100 mL of frozen resuscitation cord blood (sample 2, the cord blood is provided by the Obstetrics and Gynecology Department of the Armed Police General Hospital, and approved by the hospital ethics committee) are taken, and 100 mL of PBS (formulation: 8.0 g of NaCl, 0.2 g of KCl, 1.44 g of Na₂HPO₄, and 0.24 g of KH₂PO₄, adding distilled water to 1000 mL, and adjusting the pH to 7.4) is added to dilute the cord blood.

In sub-step 1.2, 20 mL of lymphocyte separation solution (the same name product of Beijing Jing-Meng Stem Cell Technology Co., Ltd., the same as sample density separation solution in medical device with record No.: Jinghai Machinery Equipment No. 20150002) is respectively added to ten centrifuge tubes each of a volume of 50 mL. The diluted cord blood is slowly added from above to the separation solution surface respectively, that is, 20 mL of diluted cord blood is added to each of the centrifuge tubes to maintain a clear interface between the two liquids.

In sub-step 1.3, centrifugation is performed for 30 minutes at a room temperature and with a centrifugal force of 980 g.

In sub-step 1.4, after the centrifugation, the solution in the centrifuge tube is divided into four layers with clear interfaces from top to bottom. The four layers are sequentially a light yellow plasma layer, a white membrane-like mononuclear cell layer, a transparent separation solution layer and a red erythrocyte layer from top to bottom. The white membrane-like mononuclear cell layer is carefully aspirated and put into another centrifuge tube, and is washed with PBS for 2 to 3 times, to obtain mononuclear cells.

In step 2), the cord blood NK cells are activatedly cultured.

The density of mononuclear cells is adjusted to 2×10⁶/mL (or any density ranging from 0.5×10⁶/mL to 5×10⁶/mL, and preferably ranging from 1×10⁶/mL to 3×10⁶/mL, wherein being less than 0.5×10⁶/mL or greater than 5×10⁶/mL is not advantageous for the activation of the NK cells) using the AIM V® Medium CTS™ lymphocyte culturing medium (purchased from Life Technology Company, USA). Mononuclear cell suspension is inoculated into a cell culture flask. Then, zoledronic acid (Zetai, Novartis Pharmaceuticals) with the concentration of 2 μg/mL (or any concentration ranging from 1 μg/mL to 10 μg/mL, and preferably ranging from 1 μg/mL to 5 μg/mL; the activation of the NK cells is affected if the concentration is less than 1 μg/mL and is only slightly affected if the concentration is greater than 10 μg/mL) and recombinant human interleukin-2 (De Lusheng, Beijing SiHuanShengWu) with the concentration of 1000 IU/mL (or any concentration ranging from 200 IU/mL to 2000 IU/mL, and preferably ranging from 500 IU/mL to 1500 IU/mL; the activation of the NK cells is affected if the concentration is less than 200 IU/mL, and the cells are prone to death if the concentration is greater than 2000 IU/mL) are added into the AIM V® Medium CTS™ lymphocyte culturing medium. Culturing is performed for 3 days (or any time duration ranging from 1 day to 5 days, preferably ranging from 2 days to 4 days; the activation of the NK cells is low if the culturing is performed for less than 1 day, and the activation of the NK cells is only slightly affected if the culturing is performed for more than 5 days) in an environment with a temperature of 37° C. (or any temperature ranging from 36° C. to 38° C.; the cells can not normally grow if the temperature is lower than 36° C. or higher than 38° C.) and CO₂ saturated humidity of 5%.

In step 3), the cord blood NK cells are proliferatedly cultured.

The cell fluid is pipetted to a centrifuge tube. Centrifugation is performed for 10 minutes with a centrifugal force of 150 g. The supernatant is discarded. The density of the cells is adjusted to 1×10⁶/mL (or any density ranging from 0.5×10⁶ to 5×10⁶/mL, preferably from 0.5×10⁶ to 2×10⁶/mL, wherein being less than 0.5×10⁶/mL or greater than 5×10⁶/mL is not advantageous for the proliferation of the NK cells) using the GMP S&XFM™-CD lymphocyte culturing medium (the same name product of Beijing Jing-Meng Stem Cell Technology Co., Ltd., the same as cell culture medium of medical device with record No.: Jinghai Machinery Equipment No. 20150008). The cell fluid is inoculated to a cell culture flask. Recombinant human interleukin-2 with the concentration of 1000 IU/mL (or any concentration ranging from 200 IU/mL to 2000 IU/mL, preferably ranging from 500 IU/mL to 1500 IU/mL; the proliferation of the NK cells is affected if the concentration is less than 200 IU/mL, and the cells are prone to death if the concentration is greater than 2000 IU/mL) is added. Culturing is performed in an environment with a temperature of 37° C. (or any temperature ranging from 36° C. to 38° C.; the cells can not normally grow if the temperature is lower than 36° C. or higher than 38° C.) and CO₂ saturated humidity of 5%. Fresh lymphocyte culturing medium is replenished with every other 2 to 3 days to adjust the density of the cells to a value ranging from 0.5×10⁶/mL to 5×10⁶/mL (more preferably 1×10⁶/mL, and preferably ranging from 0.5×10⁶/mL to 2×10⁶/mL; the proliferation of the cells is slow if the density of the cells is less than 0.5×10⁶/mL, and a contact inhibition is easy to form quickly for the cells, which affects the proliferation of the cells, if the density of the cells is greater than 5×10⁶/mL). Recombinant human interleukin-2 with the concentration of 1000 IU/mL (or any concentration ranging from 200 IU/mL to 2000 IU/mL, and preferably from 500 IU/mL to 1500 IU/mL) is added. Culturing is performed for 14 to 35 days (preferably 18 to 24 days, and most preferably 21 days) to obtain the cord blood NK cells. At the 21^{th} day, the number of the expanded cells is large and the killing activity of the cells is the greatest. Therefore, after the 21^{th} day, the cells enters a steady phase and the killing activity of the cells is decreased. Therefore, the 21th day is preferably taken as a harvesting point for the cord blood NK cells.

II: detection of the ex vivo expanded cord blood NK cells

1: counting the cord blood NK cells, including:

1) gently blowing the cord blood NK cell suspension, to form NK mononuclear cell suspension;

2) taking 20 μl of the NK mononuclear cell suspension, adding the NK mononuclear cell suspension into 20 μl of 0.2% trypan blue staining solution (purchased from SIGMA Company), suctioning repeatedly and mixing gently so that the mixture is uniform;

3) taking 20 μl of the mixture and adding the taken mixture into a counting plate groove of a Countstar (IC1000) cell counter (purchased from Shanghai Ruiyu Biotechnology Co., Ltd.); and

4) standing still for 1 minute and reading a measurement.

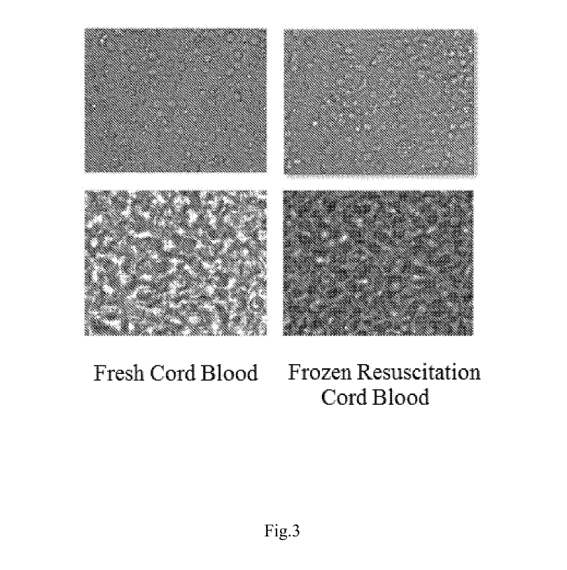

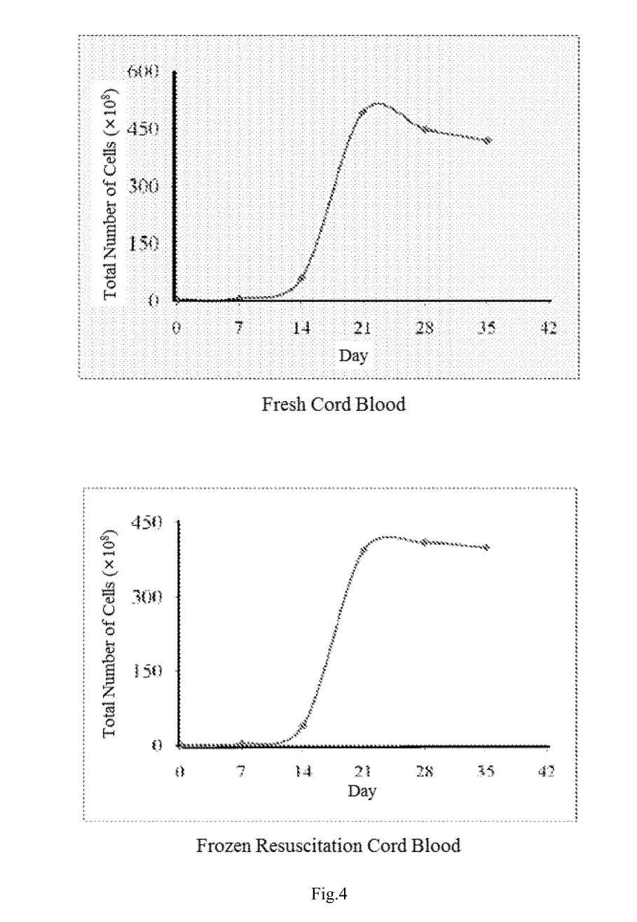

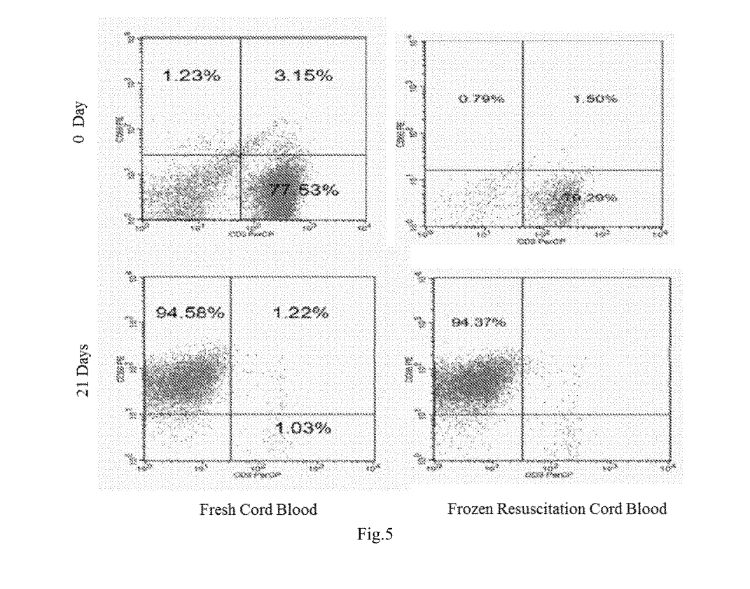

Results: after the ex vivo activating and expansion culturing of the cord blood NK cells separated from the fresh anticoagulation cord blood or frozen resuscitation cord blood (obtained by the above method in I), by observing with a microscope, the cell morphology is gradually changed from the circle shape of different sizes before the expansion to uniform and irregular cell morphology, and the volume of the cell body and the volume of the cell nucleus are increased (the cells expanded and cultured for 21 days are shown in FIG. 3). The number of the cells is proliferated from the original 4.36×10⁸ /mL (fresh cord blood) and 3.25×10⁸/mL (frozen resuscitation cord blood) to 5.08×10¹⁰/mL (fresh cord blood) and 3.94×10¹⁰/mL (frozen resuscitation cord blood) after 21 days of expansion and culturing, as shown in FIG. 4. It can also be seen from FIG. 4 that the period from the 14^{th} to the 21^{th} expanding and culturing day is a rapid proliferation phase for cells, and the period from 21^{th} day to the 35^{th} day is a slow declining phase. Therefore, the cord blood NK cells can be harvested in the period from the 14^{th} day to the 35^{th} days as required.

2: detecting the purity of the cord blood NK cells using a flow cytometer, including:

1) taking mononuclear cell suspension and centrifuging for 10 minutes with a centrifugal force of 150 g;

2) resuspending the cell precipitation with PBS, adjusting the concentration of the cells to 1×10⁶/100 μl, and placing the solution into a flow-type detection tube;

3) adding antibodies CD56 and CD3 (purchased from BD company), gently blowing and uniformly mixing the solution, incubating for 30 minutes at 4° C. in the dark, and meanwhile setting an isotype control;

4) centrifuging at 1500 g for 5 minutes and discarding the supernatant; and

5) adding 100 μl of PBS, gently blowing and uniformly mixing, and putting the solution on a machine for detection.

Results: the purity of the NK cells expanded and cultured for 21 days in I is increased from the original 1.23% (fresh cord blood) and 0.79% (frozen resuscitation cord blood) to 94.58% (fresh cord blood) and 94.37% (frozen resuscitation cord blood) respectively, as shown in FIG. 5.

3: measuring a tumoricidal activity of the cord blood NK cells with a CCK-8 method, including:

1) taking well-grown A549 cells (human lung adenocarcinoma cells, derived from ATCC) as target cells, and digesting with 0.25% trypsin;

2) performing a trypan blue staining counting, and adjusting the concentration of the cells to 5×10⁴/mL;

3) adding 100 ul per well in a 96-well plate and placing the plate in a 37° C. and 5% CO₂ environment for incubating overnight;

4) taking cord blood NK cell suspension as effector cells, and adjusting the concentration of the cells to 2×10⁶/mL;

5) adding into the 96-well plate based on an effective target ratio of 5:1, 10:1 and 20:1 in triplicate for each group;

6) culturing for 4 h in an incubator with a temperature of 37° C. and 5% CO_{2;}

7) adding 15 μl of CCK-8 (purchased from Biyuntian) to each well and continuing incubating for 2 h;

8) detecting an OD value of a wavelength of 450 nm with a microplate reader; and

9) calculating a kill rate:

Kill rate (%)=[1−(OD value of experimental group−OD value of individual effector cells)±OD value of individual target cells]×100%.

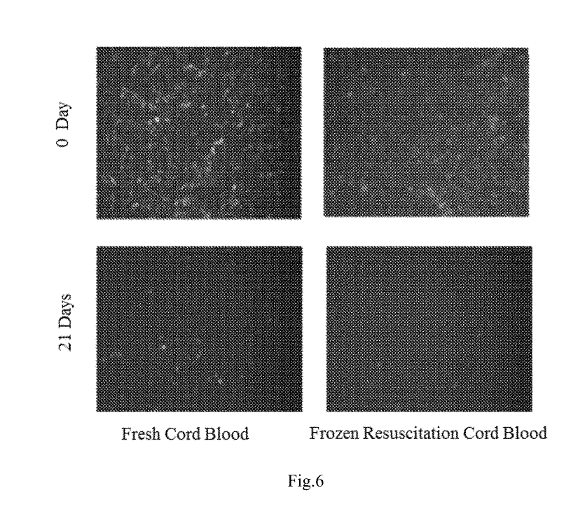

Results: it is observed under the fluorescence microscope that, the killing ability of the cord blood NK cells after 21 days of ex vivo expansion and culturing in I to A549 tumor cells is improved greatly, as shown in FIG. 6.

The above detection results show that the cord blood NK cells expanded ex vivo according to the method of the present disclosure has a large quantity, high purity, and a high killing activity to tumor cells.

Second embodiment: ex vivo expansion of cord blood NK cells (the second activatedly culturing scheme)

As shown in FIG. 1, the ex vivo expansion of the cord blood NK cells includes the following steps.

In step 1), cord blood mononuclear cells are separated from frozen resuscitation cord blood.

10 ml of frozen resuscitation cord blood (sample 3) is taken by using the same method as the first embodiment.

In step 2), the cord blood NK cells are activatedly cultured (a control group is the first embodiment).

The density of the mononuclear cells is adjusted to 2×10⁶/mL (or any density ranging from 0.5×10⁶/mL to 5×10⁶/mL, and preferably from 1×10⁶/mL to 3×10⁶/mL) using the GMP S&XFM™-CD lymphocyte culturing medium (the same name product of Beijing Jing-Meng Stem Cell Technology Co., Ltd., the same as the cell culture medium of medical device with record No.: Jinghai Machinery Equipment 20150008). Cell suspension is inoculated into a cell culture flask, and then zoledronic acid (Zetai, Novartis Pharmaceuticals) with the concentration of 2 μg/mL (or any concentration ranging from 1 μg/mL to 10 μg/mL, and preferably ranging from 1 μg/mL to 5 μg/mL) and recombinant human interleukin-2 (De Lusheng, Beijing SiHuanShengWu) with the concentration of 1000 IU/mL (or any concentration ranging from 200 IU/mL to 2000 IU/mL, and preferably ranging from 500 IU/mL to 1500 IU/mL) are added into the GMP S&XFM™-CD lymphocyte culturing medium. Culturing is performed for 1 to 5 days (preferably 2 to 4 days, and most preferably 3 days; the activation of the NK cells is low if the culturing is performed for less than 1 day, and the activation of the NK cells is only slightly affected if the culturing is performed for more than 5 days) in an environment with a temperature of 37° C. (or any temperature ranging from 36° C. to 38° C.) and CO₂ saturated humidity of 5%.

The effect that the same added reagent has on the cell activation is the same as that described in the first embodiment, and is not repeatedly described hereinafter.

In step 3), the cord blood NK cells are proliferatedly cultured.

This step is the same as that in the first embodiment.

Third embodiment: ex vivo expansion and detection of cord blood NK cells (the third activatedly culturing scheme).

As shown in FIG. 1, the ex vivo expansion of the cord blood NK cells includes the following steps.

In step 1), cord blood mononuclear cells are separated from frozen resuscitation cord blood.

10 ml of frozen resuscitation cord blood (sample 3) is taken using the same method as the first embodiment.

In step 2), the cord blood NK cells are activatedly cultured.

The density of the mononuclear cells is adjusted to 2×10⁶/mL (or any density ranging from 0.5×10⁶ to 5×10⁶/mL, and preferably from 1×10⁶ to 3×10⁶/mL) using the GMP S&XFM™-CD lymphocyte culturing medium (the same name product of Beijing Jing-Meng Stem Cell Technology Co., Ltd., the same as the cell culture medium of medical device with record No.: Jinghai Machinery Equipment 20150008). Cell suspension is inoculated into a cell culture flask. Then, zoledronic acid (Zetai, Novartis Pharmaceuticals) with the concentration of 2 μg/mL (or any concentration ranging from 1 μg/mL to 10 μg/mL, and preferably ranging from 1 μg/mL to 5 μg/mL), recombinant human interleukin-2 (De Lusheng, Beijing SiHuanShengWu) with the concentration of 1000 IU/mL (or any concentration ranging from 200 IU/mL to 2000 IU/mL, and preferably ranging from 500 IU/mL to 1500 IU/mL), recombinant human interleukin-15 (purchased from Peprotech Company) with the concentration of 10 ng/mL (or any concentration ranging from 1 ng/mL to 100 ng/mL, and preferably from 1 ng/mL to 20 ng/mL; there is no effect on the activation of the NK cells if the concentration is less than 1 ng/mL, and the activation of the NK cells is only slightly affected if the concentration is greater than 100 ng/mL) and recombinant human interleukin-18 (purchased from Peprotech Company) with the concentration of 10 ng/mL (or any concentration ranging from 1 ng/mL to 100 ng/mL, and preferably from 1 ng/mL to 20 ng/mL; there is no effect on the activation of the NK cells if the concentration is less than 1 ng/mL, and the activation of the NK cells is only slightly affected if the concentration is greater than 100 ng/mL) are added into the GMP S&XFM™-CD lymphocyte culturing medium. Culturing is performed for 3 days (or any time duration ranging from 1 to 5 days, preferably from 2 to 4 days; the activation of the NK cells is low if the culturing is performed for less than 1 day, and the activation of the NK cells is only slightly affected if the culturing is performed for more than 5 days) in an environment with a temperature of 37° C. (or any temperature ranging from 36° C. to 38° C.) and CO₂ saturated humidity of 5%.

The effect that the same added reagent has on the cell activation is the same as that described in the first embodiment, and is not repeatedly described hereinafter.

In step 3), the cord blood NK cells are proliferatedly cultured.

This step is the same as that in the first embodiment.

Fourth embodiment: ex vivo expansion of cord blood NK cells (the fourth activatedly culturing scheme).

As shown in FIG. 1, the ex vivo expansion of the cord blood NK cells includes the following steps.

In step 1), cord blood mononuclear cells are separated from frozen resuscitation cord blood.

10 ml of frozen resuscitation cord blood (sample 3) is taken using the same method as the first embodiment.

In step 2), the cord blood NK cells are activatedly cultured.

The activation culturing medium which is same as that used in the third activatedly culturing scheme is used, and the culturing condition is changed to: culturing for 1 day (or any time duration ranging from 0.5 to 3 days; the activation of the NK cells is low if the culturing is performed for less than 0.5 day, and the NK cells may be prone to death if the culturing is performed for more than 3 days) in an environment with a temperature of 39° C.±0.5° C. (or any temperature ranging from 38° C. to 40° C.; with the increased temperature, on one hand, it is advantageous for accelerating the activation of the cord blood NK cells and improving the activation efficiency, and on the other hand, the expansion amplification, purity and biological activity of the cord blood NK cells may be improved; however, if the temperature is higher than 40° C., the NK cells cannot adapt and may die) and CO₂ saturated humidity of 5%.

In step 3), the cord blood NK cells are proliferatedly cultured.

This step is the same as that in the first embodiment.

A detection is performed on the cord cell NK cells obtained in the methods according to the above embodiments.

Cord Blood NK Cell Detection

The cord blood NK cells obtained by ex vivo expanding 10 ml of frozen resuscitation cord blood (sample 3) is detected using the method described in part II of the first embodiment (a parallel operation is performed by taking sample 3 as a starting blood source and using the methods according to the first to fourth embodiments).

1: counting the cord blood NK cells

After the cord blood mononuclear cells separated from the frozen resuscitation cord blood are expanded and cultured for 21 days ex vivo, the cell expansion results are shown in Table 1-1 and Table 1-2.

In the present disclosure, NK cells with a high purity can be obtained by the two steps of activating and expanding the cord blood mononuclear cells (here, the proportion of NK cells in the mononuclear cells is low, see Table 2, and the proportion of 0-day NK cells is only 9.56%). Table 1-1 shows the total numbers of cells (including expanded NK cells and other cells) counted before and after the expansion. The absolute number of the NK cells is a value obtained by multiplying the total number of the cells and the proportion of the NK cells, that is, data of Table 1-1×Table 2=Table 1-2.

The 0-day cord blood mononuclear cells are cells before activation and expansion according to the present disclosure (including a small quantity of NK cells and other cells), and can be taken as a control; the 0-day cord blood NK cells refer to NK cells therein. The 21-day cord blood NK cell are the total number of cells after the mononuclear cells are expanded for 21 days. In this way, since the purity of the NK cells is high and most of the cells are NK cells, the cells obtained by the 21 days of expansion are called NK cells (precisely, mononuclear cells mainly composed of NK cells).

It can be seen from the results that, through all of the first embodiment to the fourth embodiment, the total number of the cord blood cells is expanded by a hundredfold (Table 1-1), the absolute number of the NK cells is expanded by a thousandfold (Table 1-2), and a comparison of the expansion effects is: the fourth embodiment>the third embodiment>the second embodiment>the first embodiment.

TABLE 1-1
Total number of cord blood cells and expansion
amplification (10 ml of cord blood)

| | First | Second | Third | Fourth |
|---|---|---|---|---|
| | embodi- | embodi- | embodi- | embodi- |
| | ment | ment | ment | ment |
| Total number of 0-day cord | 3.74 | 3.74 | 3.74 | 3.74 |
| blood mononuclear cells (×10⁷) | | | | |
| Total number of 21-day | 425 | 536 | 659 | 782 |
| expansion cells (×10⁷) | | | | |
| Expansion amplification of the | 113.64 | 143.32 | 176.20 | 209.09 |
| total number of cells | | | | |

TABLE 1-2
Absolute number of cord blood NK cells and expansion
amplification (10 ml of cord blood)

| | First | Second | Third | Fourth |
|---|---|---|---|---|
| | embodi- | embodi- | embodi- | embodi- |
| | ment | ment | ment | ment |
| Absolute number of 0-day cord | 3.58 | 3.58 | 3.58 | 3.58 |
| blood NK cells (×10⁶) | | | | |
| Absolute number of 21-day cord | 3854.3 | 4955.9 | 6178.1 | 7459.5 |
| blood NK cells (×10⁶) | | | | |
| Expansion amplification of cord | 1078.00 | 1386.08 | 1727.93 | 2086.32 |
| blood NK cells | | | | |

2: detecting the purity of the cord blood NK cells by using a flow cytometer.

NK cell purity results obtained by activating and expanding the cord blood mononuclear cells ex vivo for 21 days are shown in Table 2, where the cord blood mononuclear cells are separated from the frozen resuscitation cord blood.

TABLE 2
Purities of cord blood NK cells

| | First | Second | Third | Fourth |
|---|---|---|---|---|
| | embodi- | embodi- | embodi- | embodi- |
| | ment | ment | ment | ment |
| Purity of 0-day cord blood NK | 9.56% | 9.56% | 9.56% | 9.56% |
| cells | | | | |
| Purity of 21-day cord blood NK | 90.69% | 92.46% | 93.75% | 95.39% |
| cells | | | | |

The data in Table 2 indicates that, after the expansions in the above embodiments, the proportions of NK cells in the cord blood mononuclear cells are all improved from the original 9.56% to the purities of the NK cells greater than or equal to 90%. The purities obtained by the first embodiment to the fourth embodiment have the following relationship: fourth embodiment>third embodiment>second embodiment>first embodiment.

3: measuring a tumoricidal activity of the cord blood NK cells with a CCK-8 method.

The experiment takes 0-day cord blood mononuclear cells (cells before activation and expansion) as a negative control, and takes CIK cells (an immune cell that kills tumors) as a positive control. For the culturing of the CIK cells, reference may be made to a Reference Document (Document 6: Adoptive immunotherapy with cytokine-induced killer cells generated with a new good manufacturing practice-grade protocol. Cytotherapy, 2012; Early Online: 1-10. DOI: 10.3109/14653249.2012.681038).

The results are shown in Table 3. By observing under a fluorescence microscope, it is shown that the cord blood NK cells harvested after 21 days of ex vivo expansion according to the embodiments all have a strong killing ability to A549 tumor cells, and as compared with the positive control CIK cells, the NK cells obtained after the expansion according to the present disclosure are more active. The killing abilities of the four embodiments have the following relationship under the same effective target ratio: fourth embodiment>third embodiment>second embodiment>first embodiment. Therefore, the cells expanded according to the fourth embodiment have the strongest cell killing ability.

TABLE 3
Killing ability of cord blood NK cells to A549 tumor cells

| | | First | Second | Third | Fourth | |
|---|---|---|---|---|---|---|
| | | embodi- | embodi- | embodi- | embodi- | |
| | | ment | ment | ment | ment | |
| | 0 | (21 | (21 | (21 | (21 | CIK |
| | day | days) | days) | days) | days) | cell |
| Effective target | 9.71% | 52.18% | 54.21% | 77.52% | 79.11% | 30.56% |
| ratio (5:1) | | | | | | |
| Effective target | 17.42% | 60.77% | 63.85% | 85.63% | 87.27% | 42.78% |
| ratio (10:1) | | | | | | |
| Effective target | 23.74% | 72.90% | 71.53% | 88.91% | 91.45% | 55.61% |
| ratio (20:1) | | | | | | |

It can be seen from the above embodiments that, the ex vivo expansion method for the cord blood NK cells according to the present disclosure has a simple operation and is safe, and comparison results between the method according to the present disclosure and the existing methods for separating and expanding the cord blood NK cells (the prior art) are shown in Table 4.

TABLE 4
Comparison between the conventional technologies and technologies according to the present disclosure

| | | | | | | | Cellular properties | |
|---|---|---|---|---|---|---|---|---|
| | | | | Related products | | | | Yield |
| | Technology | | | Sorting | Medium and | trophoblast | | (per part of |
| | Cost | Versatility | Safety | reagent | its additives | layer | Purity | cord blood) |
| Celgene | High | Poor | Poor | NK negative | IMDM, fetal | K562 | >80% | 10⁹⁻¹⁰ |
| | | | | selection kit | bovine serum, | pretreated | | |
| | | | | | IL-2, PHA-P | with mitomycin C | | |
| Glycostem | High | Poor | Not | CD34 positive | GBGM, 10% | No need | >90% | 10⁹⁻¹⁰ |
| | | | bad | selection kit | human serum, 12 | | | |
| | | | | | kinds of cytokines | | | |
| The present | Low | Good | Good | No need | Lymphocyte culturing | No need | >90% | 10¹⁰⁻¹¹ |
| disclosure | | | | | medium, IL-2, Zetai | | | |

Notes:
the amount of one part of cord blood is 100 ml (typically, the amount of one part of cord blood is in a range from 50 ml to 120 ml).

Fifth embodiment: ex vivo expansion kit for cord cell NK cells.

The ex vivo expansion kit for the cord blood NK cells according to the present disclosure mainly includes the following reagents:

1): lymphocyte separation solution of the cord blood NK cells (the same name product of Beijing Jing-Meng Stem Cell Technology Co., Ltd., the same as sample density separation solution of medical device with record No.: Jinghai Machinery Equipment No. 20150002), or other commercially available lymphocyte separation solution;

2): a cord blood NK cell activation culturing medium: an AIM V® Medium CTS™ medium (purchased from Life Technology, USA) or a GMP S&XFM™-CD lymphocyte culturing medium (the same name product of Beijing Jing-Meng Stem Cell Technology Co., Ltd., the same as cell culture medium of medical device with record No.: Jinghai Machinery Equipment No. 20150008), and zoledronic acid, recombinant human interleukin-2, recombinant human interleukin-15 and recombinant human interleukin-18; and

3): a cord blood NK cell proliferation culturing medium: a GMP S&XFM™-CD lymphocyte culturing medium (the same name product of Beijing Jing-Meng Stem Cell Technology Co., Ltd., the same as cell culture medium of medical device with record No.: Jinghai Machinery Equipment No. 20150008) and recombinant human interleukin-2.

The kit may be used by making reference to the methods according to the first to fourth embodiments.

In summary, the present disclosure has the following advantages.

1): no cell sorting is required.

2): no trophoblast cell is required.

3): operations are simple and a good versatility is achieved.

4): the cost is low.

5): the adopted reagents contain no animal-source compositions, hence a good safety is achieved.

6): the yield of NK cells is high. The purity of the obtained cord blood NK cells may be above 90%, and the total number of the cells can reach 10¹⁰⁻¹¹ cells per part of cord blood (the sample is 100 ml according to the first embodiment).

7): the killing ability to tumor cells is high.

### INDUSTRIAL APPLICABILITY

In the present disclosure, the cord blood NK cells are obtained by activatedly culturing and proliferatedly culturing separated cord blood mononuclear cells. It is not required to sort cells, and no trophoblast cells are required. The technical solutions according to the present disclosure have simple operations, a good versatility and a low cost. The technical solutions according to the present disclosure also have a good safety since the reagent contains no animal-source compositions. In addition, the yield of harvested NK cells is high, the total number of cells can reach 10¹⁰⁻¹¹ cells per part of cord blood, the purity is above 90%, and the killing ability to tumor cells is high. The present disclosure can be applied to the preparation of tumor immunotherapy drugs.

1. An ex vivo expansion method for cord blood Nature killer (NK) cells, comprising the following steps:
step 1): activatedly culturing the cord blood NK cells by: adjusting the density of cord blood mononuclear cells to 0.5×106 to 5×106/mL using a lymphocyte culturing medium, adding at least zoledronic acid with the concentration ranging from 1 μg/mL to 10 μg/mL and recombinant human interleukin-2 with the concentration ranging from 200 IU/mL to 2000 IU/mL, culturing for 1 to 5 days in an environment with a temperature ranging from 36° C. to 40° C. and CO2 saturated humidity of 5%, and collecting cell fluid; and
step 2): proliferatedly culturing the cord blood NK cells by: obtaining cells from the cell fluid collected in step 1), adjusting the density of the cells to 0.5×106 to 5×106/mL using a lymphocyte culturing medium, adding recombinant human interleukin-2 with the concentration ranging from 200 IU/mL to 2000 IU/mL, culturing in an environment with a temperature ranging from 36° C. to 38° C. and CO2 saturated humidity of 5%, replenishing with fresh culturing medium every other 2 to 3 days and adjusting the density of the cells to 0.5×106 to 5×106/mL, and culturing for 14 to 35 days to harvest the cord blood NK cells.

2. The ex vivo expansion method for the cord blood mononuclear cells according to claim 1, wherein the cord blood mononuclear cells used in step 1) are separated from fresh anticoagulation cord blood or frozen resuscitation cord blood by:
1.1: taking the fresh anticoagulation cord blood or frozen resuscitation cord blood, and diluting it with Phosphate Buffered Saline (PBS) of a volume which is 1 to 2 times the volume of the cord blood;
1.2: adding slowly the diluted cord blood from above to the lymphocyte separation solution, and maintaining a clear interface between the diluted cord blood and the lymphocyte separation solution, wherein the volume of the diluted cord blood is equal to the volume of the lymphocyte separation solution;
1.3: centrifuging for 20 to 30 minutes at a room temperature and with a centrifugal force of 980 g; and
1.4: after the centrifugation, aspirating a white membrane-like mononuclear cell layer, which is a second layer from the top, and washing the mononuclear cell layer with the PBS, to obtain cord blood mononuclear cells.

3. The ex vivo expansion method for the cord blood NK cells according to claim 1, wherein the lymphocyte culturing medium used in step 1) may be AIM V® Medium CTS™ (commercially available from Life Technology Company, USA) or GMP S&XFM™-CD lymphocyte culturing medium (the same name product of Beijing Jing-Meng Stem Cell Technology Co., Ltd.), and is preferably the GMP S&XFM™-CD lymphocyte culturing medium.

4. The ex vivo expansion method for the cord blood NK cells according to claim 3, wherein in the case that the lymphocyte culturing medium used in step 1) is the AIM V® Medium CTS™, the activated culturing preferably is: adjusting the density of the cells to 1×106 to 3×106/mL using the AIM V® Medium CTS™ lymphocyte culturing medium, adding zoledronic acid with the concentration ranging from 1 μg/mL to 5 μg/mL and recombinant human interleukin-2 with the concentration ranging from 500 IU/mL to 1500 IU/mL, and culturing for 2 to 4 days in an environment with a temperature ranging from 36° C. to 38° C. and CO2 saturated humidity of 5%; the activatedly culturing more preferably is: adjusting the density of the cells to 2×106/mL using the AIM V® Medium CTS™ lymphocyte culturing medium, adding zoledronic acid with the concentration of 2 μg/mL and recombinant human interleukin-2 with the concentration of 1000 IU/mL, and culturing for 3 days in an environment with a temperature of 37° C. and CO2 saturated humidity of 5%.

5. The ex vivo expansion method for the cord blood NK cells according to claim 3, wherein in the case that the lymphocyte culturing medium used in step 1) is the GMP S&XFM™-CD lymphocyte culturing medium, the activatedly culturing preferably is one of the followings:
A: adjusting the density of the cells to 1×106 to 3×106/mL using the GMP S&XFM™-CD lymphocyte culturing medium, adding zoledronic acid with the concentration ranging from 1 μg/mL to 5 μg/mL and recombinant human interleukin-2 with the concentration ranging from 500 IU/mL to 1500 IU/mL, and culturing for 2 to 4 days in an environment with a temperature ranging from 36° C. to 38° C. and CO2 saturated humidity of 5%; and the activatedly culturing more preferably is: adjusting the density of the cells to 2×106/mL using the GMP S&XFM™-CD lymphocyte culturing medium, adding zoledronic acid with the concentration of 2 μg/mL and recombinant human interleukin-2 with the concentration of 1000 IU/mL, and culturing for 3 days in an environment with a temperature of 37° C. and CO2 saturated humidity of 5%;
B: adjusting the density of the cells to 1×106 to 3×106/mL using the GMP S&XFM™-CD lymphocyte culturing medium, adding zoledronic acid with the concentration ranging from 1 μg/mL to 5 μg/mL, recombinant human interleukin-2 with the concentration ranging from 500 IU/mL to 1500 IU/mL, recombinant human interleukin-15 with the concentration ranging from 1 ng/mL to 100 ng/mL (preferably from 1 ng/mL to 20 ng/mL) and recombinant human interleukin-18 with the concentration ranging from 1 ng/mL to 100 ng/mL (preferably from 1 ng/mL to 20 ng/mL), and culturing for 2 to 4 days in an environment with a temperature ranging from 36° C. to 38° C. and CO2 saturated humidity of 5%; the activatedly culturing more preferably is: adjusting the density of the cells to 2×106/mL using the GMP S&XFM™-CD lymphocyte culturing medium, adding zoledronic acid with the concentration of 2 μg/mL, recombinant human interleukin-2 with the concentration of 1000 IU/mL, recombinant human interleukin-15 with the concentration of 10 ng/mL and recombinant human interleukin-18 with the concentration of 10 ng/mL, and culturing for 3 days in an environment with a temperature of 37° C. and CO2 saturated humidity of 5%; and
C: adjusting the density of the cells to 1×106 to 3×106/mL using the GMP S&XFM™-CD lymphocyte culturing medium, adding zoledronic acid with the concentration ranging from 1 μg/mL to 5 μg/mL, recombinant human interleukin-2 with the concentration ranging from 500 IU/mL to 1500 IU/mL, recombinant human interleukin-15 with the concentration ranging from 1 ng/mL to 100 ng/mL (preferably from 1 ng/mL to 20 ng/mL) and recombinant human interleukin-18 with the concentration ranging from 1 ng/mL to 100 ng/mL (preferably from 1 ng/mL to 20 ng/mL), and culturing for 0.5 to 1 day in an environment with a temperature ranging from 38.5° C. to 39.5° C. and CO2 saturated humidity of 5%; the activatedly culturing more preferably is: adjusting the density of the cells to 2×106/mL using the GMP S&XFM™-CD lymphocyte culturing medium, adding zoledronic acid with the concentration of 2 μg/mL, recombinant human interleukin-2 with the concentration of 1000 IU/mL, recombinant human interleukin-15 with the concentration of 10 ng/mL and recombinant human interleukin-18 with the concentration of 10 ng/mL, and culturing for 1 day in an environment with a temperature of 39° C. and CO2 saturated humidity of 5%.

6. The ex vivo method for the cord blood NK cells according to claim 1, wherein the lymphocyte culturing medium used in step 2) is the GMP S&XFM™-CD lymphocyte culturing medium (product of Beijing Jing-Meng Stem Cell Technology Co., Ltd.), and the proliferatedly culturing of the cord blood NK cells preferably is: pipetting the cell fluid to a centrifuge tube, centrifuging for 10 minutes with a centrifugal force of 150 g, discarding the supernatant, adjusting the density of the cells to 0.5×106 to 2×106/mL (more preferably 1×106/mL) using the GMP S&XFM™-CD lymphocyte culturing medium, adding recombinant human interleukin-2 with the concentration ranging from 500 IU/mL to 1500 IU/mL (more preferably 1000 IU/mL), culturing in an environment with a temperature of 37° C. and CO2 saturated humidity of 5%, replenishing with fresh medium every other 2 to 3 days and adjusting the density of the cells to 0.5×106 to 2×106/mL (more preferably 1×106/mL), adding recombinant human interleukin-2 with the concentration ranging from 500 IU/mL to 1500 IU/mL (more preferably 1000 IU/mL), and culturing for 18 to 24 days (more preferably 21 days), to obtain the cord blood NK cells.

7. The ex vivo expansion method for the cord blood NK cells according to claim 1, wherein a dedicated activation culturing medium used in step 1) comprises one of group A to group C:
group A: an AIM V® Medium CTS™ lymphocyte culturing medium added with zoledronic acid with the concentration ranging from 1 μg/mL to 10 μg/mL and recombinant human interleukin-2 with the concentration ranging from 200 IU/mL to 2000 IU/mL;
preferably, an AIM V® Medium CTS™ lymphocyte culturing medium added with zoledronic acid with the concentration ranging from 1 μg/mL to 5 μg/mL and recombinant human interleukin-2 with the concentration ranging from 500 IU/mL to 1500 IU/mL; and
more preferably, an AIM V® Medium CTS™ lymphocyte culturing medium added with zoledronic acid with the concentration of 2 μg/mL and recombinant human interleukin-2 with the concentration of 1000 IU/mL;
group B: a GMP S&XFM™-CD lymphocyte culturing medium added with zoledronic acid with the concentration ranging from 1 μg/mL to 10 μg/mL and recombinant human interleukin-2 with the concentration ranging from 200 IU/mL to 2000 IU/mL;
preferably, a GMP S&XFM™-CD lymphocyte culturing medium added with zoledronic acid with the concentration ranging from 1 μg/mL to 5 μg/mL and recombinant human interleukin-2 with the concentration ranging from 500 IU/mL to 1500 IU/mL; and
more preferably, a GMP S&XFM™-CD lymphocyte culturing medium added with zoledronic acid with the concentration of 2 μg/mL and recombinant human interleukin-2 with the concentration of 1000 IU/mL; or
group C: a GMP S&XFM™-CD lymphocyte culturing medium added with zoledronic acid with the concentration ranging from 1 μg/mL to 10 μg/mL, recombinant human interleukin-2 with the concentration ranging from 200 IU/mL to 2000 IU/mL, recombinant human interleukin-15 with the concentration ranging from 1 ng/mL to 100 ng/mL and recombinant human interleukin-18 with the concentration ranging from 1 ng/mL to 100 ng/mL;
preferably, a GMP S&XFM™-CD lymphocyte culturing medium added with zoledronic acid with the concentration ranging from 1 μg/mL to 5 μg/mL, recombinant human interleukin-2 with the concentration ranging from 500 IU/mL to 1500 IU/mL, recombinant human interleukin-15 with the concentration ranging from 1 ng/mL to 20 ng/mL and recombinant human interleukin-18 with the concentration ranging from 1 ng/mL to 20 ng/mL; and
more preferably, a GMP S&XFM™-CD lymphocyte culturing medium added with zoledronic acid with the concentration of 2 μg/mL, recombinant human interleukin-2 with the concentration of 1000 IU/mL, recombinant human interleukin-15 with the concentration of 10 ng/mL and recombinant human interleukin-18 with the concentration of 10 ng/mL.

8. The ex vivo expansion method for the cord blood NK cells according to claim 1, wherein a dedicated proliferation culturing medium used in step 2) is a GMP S&XFM™-CD lymphocyte culturing medium added with recombinant human interleukin-2 with the concentration ranging from 200 IU/mL to 2000 IU/mL (preferably 1000 IU/mL).

9. Cord blood NK cells with a purity above 90% harvested by using the ex vivo expansion method comprising the following steps:
step 1): activatedly culturing the cord blood NK cells by: adjusting the density of cord blood mononuclear cells to 0.5×106 to 5×106/mL using a lymphocyte culturing medium, adding at least zoledronic acid with the concentration ranging from 1 μg/mL to 10 μg/mL and recombinant human interleukin-2 with the concentration ranging from 200 IU/mL to 2000 IU/mL, culturing for 1 to 5 days in an environment with a temperature ranging from 36° C. to 40° C. and CO2 saturated humidity of 5%, and collecting cell fluid; and
step 2): proliferatedly culturing the cord blood NK cells by: obtaining cells from the cell fluid collected in step 1), adjusting the density of the cells to 0.5×106 to 5×106/mL using a lymphocyte culturing medium, adding recombinant human interleukin-2 with the concentration ranging from 200 IU/mL to 2000 IU/mL, culturing in an environment with a temperature ranging from 36° C. to 38° C. and CO2 saturated humidity of 5%, replenishing with fresh culturing medium every other 2 to 3 days and adjusting the density of the cells to 0.5×106 to 5×106/mL, and culturing for 14 to 35 days to harvest the cord blood NK cells.

10. An application of cord blood NK cells in preparing tumor immunotherapy drugs or in a tumor immunotherapy, wherein the cord blood NK cells with a purity above 90% harvested by using the ex vivo expansion method comprising the following steps:
step 1): activatedly culturing the cord blood NK cells by: adjusting the density of cord blood mononuclear cells to 0.5×106 to 5×106/mL using a lymphocyte culturing medium, adding at least zoledronic acid with the concentration ranging from 1 μg/mL to 10 μg/mL and recombinant human interleukin-2 with the concentration ranging from 200 IU/mL to 2000 IU/mL, culturing for 1 to 5 days in an environment with a temperature ranging from 36° C. to 40° C. and CO2 saturated humidity of 5%, and collecting cell fluid; and
step 2): proliferatedly culturing the cord blood NK cells by: obtaining cells from the cell fluid collected in step 1), adjusting the density of the cells to 0.5×106 to 5×106/mL using a lymphocyte culturing medium, adding recombinant human interleukin-2 with the concentration ranging from 200 IU/mL to 2000 IU/mL, culturing in an environment with a temperature ranging from 36° C. to 38° C. and CO2 saturated humidity of 5%, replenishing with fresh culturing medium every other 2 to 3 days and adjusting the density of the cells to 0.5×106 to 5×106/mL, and culturing for 14 to 35 days to harvest the cord blood NK cells.

11. An ex vivo expansion kit for cord blood NK cells, comprising:
1): lymphocyte separation solution of the cord blood NK cells: product of Beijing Jing-Meng Stem Cell Technology Co., Ltd., the same as sample density separation solution of medical device with record No.: Jinghai Machinery Equipment No. 20150002, or other commercially available lymphocyte separation solutions;
2): a cord blood NK cell activation culturing medium: an AIM V® Medium CTS™ medium (product of Life Technology, USA) or a GMP S&XFM™-CD lymphocyte culturing medium (product of Beijing Jing-Meng Stem Cell Technology Co., Ltd., the same as cell culture medium of medical device with record No.: Jinghai Machinery Equipment No. 20150008), and zoledronic acid, recombinant human interleukin-2, recombinant human interleukin-15 and recombinant human interleukin-18; and
3): a cord blood NK cell proliferation culturing medium: a GMP S&XFM™-CD lymphocyte culturing medium (product of Beijing Jing-Meng Stem Cell Technology Co., Ltd., the same as cell culture medium of medical device with record No.: Jinghai Machinery Equipment No. 20150008) and recombinant human interleukin-2.
